(19) United States
(12) Patent Application Publication
(10) Pub. No.: US 2008/0139486 A1
(43) Pub. Date: Jun. 12, 2008

(54) Flavonol-Based Medicine For Treating And/Or Preventing Endoprosthetic Restenosis And atheromatous Disease In Coronary Patients

(76) Inventors: Ramaroson Andriantsitohaina, Tierce (FR); Gerald Roul, Strasbourg (FR)

Correspondence Address: YOUNG & THOMPSON, 209 Madison Street, Suite 500, ALEXANDRIA, VA, 22314, US

(21) Appl. No.: 11/629,524

(22) PCT Filed: Jun. 15, 2005
(86) PCT No.: PCT/FR05/01505

§ 371 Date: Aug. 28, 2007

(30) Foreign Application Priority Data
Jun. 15, 2004 (FR) 0451279

(51) Int. Cl. A61K 31/35; A61P 9/00; A61K 31/7048

(52) U.S. Cl. 514/27; 514/456

(57) ABSTRACT

The invention concerns a composition for implementing a preventive therapeutic treatment method against restenosis, characterized in that it comprises 60 to 80 wt. % of compounds of the flavonol family. The composition is useful for implementing a preventive therapeutic treatment method against restenosis and atheromatous disease in coronary patients.

The invention has as its object a new medication as well as its use in the prevention of restenosis and atheromatous illness in coronary patients.

Heart disease is a real public health problem in all developed countries. Following the works of Gruëntzig, coronary angioplasty, notwithstanding its palliative nature, is indispensable as a reference therapeutic method. This method has been greatly improved by the advent of endoprostheses (stent, in English).

However, although the endoprosthesis has proven its effectiveness in the monitoring of initial complications and for the prevention of the restenosis of balloon angioplasty, the endoprosthetic restenosis (“intra stent” in English), secondary to the exaggerated hyperplasia of the smooth muscle cells, nevertheless still remains a significant complication and its preventive treatment still remains disappointing.

This is why the inventors are set on developing a therapeutic solution that makes it possible to modulate this hyperplasia without, however, preventing it.

This is why, according to a first aspect, the invention has as its object a composition for the implementation of a preventive therapeutic method of treatment against the endoprosthetic restenosis and against the atheromatous illness in coronary patients, characterized in that it comprises 60% and 80% by weight of compounds of the flavonol family.

Within the scope of this invention, flavonols are defined as derivative compounds of the following monomeric structures of formula (I):

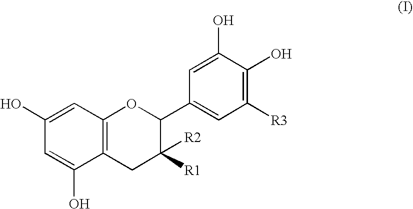

that are defined by:
(+)-catechin, when R1=OH, R2=H, and R3=H.
(−)-epicatechin, when R1=H, R2=OH, and R3=H,
(+)-gallocatechin, when R1=OH, R2=H, and R3=OH,
(−)-epigallocatechin, when R1=H, R2=OH, and R3=OH.

The object of the invention is more particularly a composition as defined above, characterized in that it comprises, expressed in terms of percentages of flavonols that are present:
0% to 20% by weight of catechin and/or catechin-SH,
45% to 75% by weight of epicatechin and/or epicatechin-SH,
0% to 15% by weight of epicatechin gallate and/or epicatechin gallate-SH, and
15% to 25% by weight of epigallocatechin-SH.

The composition as defined above can also comprise between 10% and 30% by weight of compounds of the family of anthocyans.

Within the scope of this invention, anthocyans are defined as mono- or polyglucosylated compounds whose aglycons (anthocyanidins) have the following structures of formula (II):

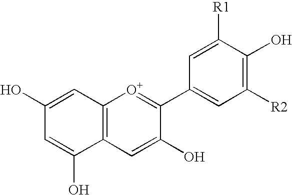

that are defined by:

Malvidin, when R1 and R2 each represent a methoxy radical,

Peonidin, when R1 represents a methoxy radical and R2 represents a hydrogen atom,

Delphinidin, when R1 and R2 each represent a hydroxy radical, and

Petunidin, when R1 represents a methoxy radical and R2 represents a hydroxy radical.

The object of the invention is more particularly a composition as defined above, characterized in that it comprises, expressed by percentages of anthocyans that are present:
55% to 100% by weight of malvidin-3-O-glucoside, acetylated malvidin-3-O-glucoside and/or coumarylated malvidin-3-O-glucoside,
0% to 30% by weight of peonidin-3-O-glucoside, acetylated peonidin-3-O-glucoside and/or coumarylated peonidin-3-O-glucoside,
0% to 20% by weight of petunidin-3-O-glucoside and/or acetylated petunidin-3-O-glucoside,
0% to 10% by weight of cyanidin 3-O-gluoside and/or acetylated petunidin-3-O-glucoside, and
0% to 10% by weight of delphinidin 3-O-glucoside and/or acetylated delphinidin-3-O-glucoside.

The composition as defined above can also comprise between 10% and 30% by weight of compounds of the family of phenolic acids.

As phenolic acids that are suitable to this invention, there are more particularly:

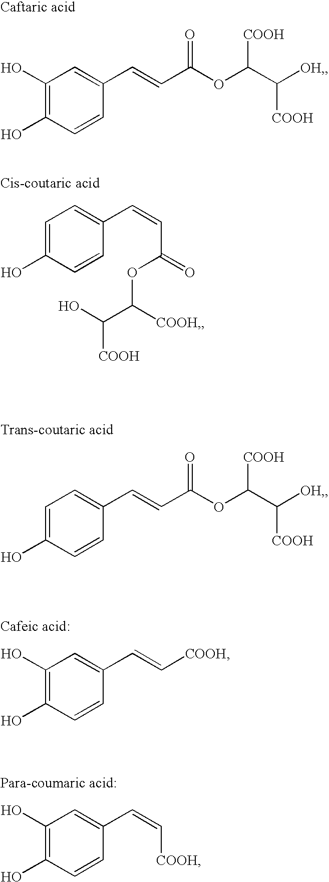

or the GRP (2-S-glutathionyl caftaric acid, tartaric acid ester).

The composition as defined above more particularly comprises, expressed in terms of percentages of phenolic acids that are present:
20% to 25% by weight of caftaric acid,
30% to 40% by weight of cafeic acid,
15% to 25% by weight of trans-coutaric acid,
15% to 25% by weight of para-coumaric acid,
optionally up to 5% of cis-coutaric acid, and
optionally up to 5% of GRP.

The composition as defined above can also comprise up to 5% by weight of compounds of the family of flavonols or their glucosylated derivatives.

Flavonols are defined more particularly as the compounds of formula (III):

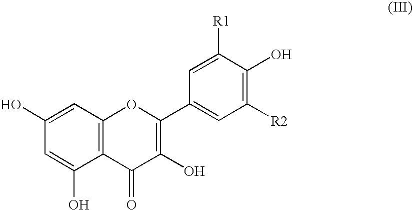

that are defined by:

Kaempferol, when R1 and R2 each represent a hydrogen atom,

Quercetin, when R1 represents a hydroxy radical, and R2 represents a hydrogen atom,

Myricetin, when R1 and R2 each represent a hydroxy radical, and

Isorhametin, when R1 represents a methoxy radical and R2 represents a hydrogen atom.

The object of the invention is more particularly a composition as defined above, characterized in that it comprises, expressed in terms of percentage of flavonols that are present:
15% to 35% by weight of myricetol glucoside,
15% to 35% by weight of quercetol,
10% to 25% by weight of myricitol and
15% to 30% by weight of quercetol glucoside.

The object of the invention is preferably a composition as defined above, characterized in that it is obtained by a process that comprises the following successive stages:
A stage (a) for red wine distillation,
A stage (b) for concentration of the distillate that is obtained in stage (a), and
A stage (c) for drying the concentrate that is obtained in stage (b).

The composition is thus obtained in the form of a powder that generally contains at most 5% by weight of moisture.

It can be formulated, for example, in capsule or tablet form by incorporating, if necessary or if desired, one or more excipients that are generally used in this type of galenical form. It may also involve bags of water-soluble granules.

According to another aspect, the object of the invention is the application of a composition as defined above for the implementation of a method for preventive therapeutic treatment against endoprosthetic restenosis and against atheromatous illness in coronary patients.

The medicinal dose that is to be absorbed by the patient is between about 0.005 gram of active ingredient per day and per kilogram and 0.025 gram of active ingredient of composition per day and per kilogram. For a patient weighing 80 kg, the more particularly recommended daily dose of active ingredient is between 0.5 g/day and 1.5 g/day.

The following examples illustrate the invention without, however, limiting it.

### A)—Preparation of a Composition According to the Invention

A composition (composition A) according to the invention is prepared via extraction by distilling Burgundy red wine (grape variety: Cabernet-Sauvignon), by concentrating the thus obtained distillate, then by drying the concentrate. The powder that is obtained contains about 5% by weight of moisture. After dissolving at 0.13% by weight in a hydro-alcoholic solution (alcohol level: 12% (v/v)), a solution is obtained that contains about:
200 mg/liter of phenolic acids, including about:
   70 mg/l of cafeic acid,
   50 mg/l of caftaric acid,
   40 mg/l of trans-coutaric acid, and
   30 mg/l of para-coumaric acid;
250 mg/l of anthocyans, including about:
   125 mg/l of malvidin-3-O-glucoside,
   40 mg/l of acetylated malvidin-3-O-glucoside,
   20 mg/l of coumarylated malvidin-3-O-glucoside,
   15 mg/l of peonidin-3-O-glucoside,
   15 mg/l of petunidin-3-O-glucoside, and
   10 mg/l of delphinidin-3-O-glucoside;
20 mg/l of flavonols, including about:
   5 mg/l of myricetol glucoside,
   5 mg/l of quercetol glucoside,
   5 mg/l of myricetol, and
   5 mg/l of quercetol;
900 mg/l of flavonols, including about:
   500 mg/l of epicatechin-SH,
   190 mg/l of epigallocatechin-SH,
   70 mg/l of epicatechin gallate-SH,
   70 mg/l of catechin-SH,
   60 mg/l of catechin, and
   20 mg/l of epicatechin.

### B)—Evaluation of the Effect of Composition (A) According to the Invention for Preventing “Endoprosthetic” Restenosis in Rabbits.

The objective of the study was to evaluate the influence of the composition (A) on the restenosis that is induced by the implantation of a “vascular endoprosthesis” for four weeks in the iliac arteries of rabbits.

### Experimental Protocol

For 6 weeks, 11 New Zealand male rabbits of 3 to 4 kg were subjected to a hypercholesterolemic regimen in which 1% cholesterol was added relative to the standard regimen.

At the end of this period, a vascular endoprosthesis was installed in the right iliac artery via the right carotid access (intervention carried out in Rangueil experimental surgery). When the conditions allowed it (procedure time, vital constants of animals), implantation in the left iliac artery was also carried out.

The implantation of the vascular endoprosthesis was systematically preceded by a balloon predilation, and (inflation to 10 atmospheres for 1 minute) then done by two inflations of 30 seconds each to 10 atmospheres. The procedure was carried out under fluoroscopic monitoring and under anesthesia (Rompun+Imalgene). Before the operation, the animals received heparin (500 U/kg), aspirin (50 mg) and Corvasal (0.3 mg) in bolus IV.

Upon awakening, the animals profited from a daily intramuscular injection of antibiotic for 5 days. For 28 days, all of the animals received a hypercholesterolemic regimen in which 0.2% cholesterol was added relative to the standard regimen and a treatment with aspirin and with Plavix (7.5 mg of each molecule dissolved in drinking water).

After intervention, the animals were sorted in such a way as to take advantage of treatment with composition A in a way that was blind to the experimenter.

This composition A is administered dissolved in drinking water with the posology of 30 mg/kg. Below, the group A (5 treated animals) represents the group of treated animals, and the group B (6 animals) represents the placebo group. 16 iliac arteries were implanted, eight in each group.

28 days after the installation of the endoprostheses, the animals are sacrificed under general anesthesia, whereby the vessels were fixed in vivo by injection of paraformaldehdye (buffered at 10%) after bleeding and washing with PBS buffer. The instrumented iliac arteries and the entire aorta are sampled and kept at ambient temperature.

### Identification of the Samples

11 rabbits were operated on and treated as indicated above for a blind histological and histomorphometric evaluation.

Three left iliac arteries and five right iliac arteries were removed from group A of 5 rabbits (treated with composition A).

Five samples of left iliac arteries and three right iliac arteries were removed from the group B of 6 rabbits (placebo).

Each of the sampled arteries is identified and divided into sections as indicated in Table 1 below:

TABLE 1

| | | | | |
|---|---|---|---|---|
| Identification | Length of the | | Length of the | |
| of | Left Iliac | Number of | Right Iliac | Number of |
| the Animal | Artery (in mm) | Sections | Artery (in mm) | Sections |

Group A

| | | | | |
|---|---|---|---|---|
| 2.1 FXD | 33 | 4 | 8 | 2 |
| 6 FUN | 13 | 2 | 16 | 3 |
| 11.1 FUO | 13 | 2 | 20 | 3 |
| 16 FIR | — | — | 8 | 2 |
| 20 FUY | — | — | 8 | 2 |
| Subtotals | 3 | 8 | 5 | 12 |

Group B

| | | | | |
|---|---|---|---|---|
| 3.1 FXH | 9 | 2 | 12 | 2 |
| 4 FW | 18 | 3 | 18 | 3 |
| 5.1 FBZ | 25 | 4 | — | — |
| 8.1 FUF | 9 | 2 | — | — |
| 9. FUN | — | — | 16 | 3 |
| 12 FUG | 13 | 2 | — | — |
| Subtotals | 5 | 13 | 3 | 8 |
| Totals | 8 | 21 | 8 | 20 |

### Preparation of Histopathological Samples

The sampled arteries are dehydrated by means of alcoholic solutions of increasing concentrations. They are clarified with xylene and then incorporated in methyl poly(methacrylate) (PMMA). The sections of each artery are obtained by microcutting techniques (microcutting in English) and grinding techniques that are adapted to those described by Donath (Donath, K.; Brunner, G.: “A Method for the Study of Undecalcified Bone and Teeth with Attached Soft Tissues.” J. Oral. Pathol., 11; 318-326, 1982).

The sections are colored with modified Paragon to allow qualitative and quantitative analyses.

### Interpretation

The histological slides are examined according to a blind analysis by light microscopy (NIKON™ Eclipse E600 microscope, mounted with 4×, 10×, 20× and 40× magnifying lenses, linked to a DN 100 NIKON™ photo device).

The semi-quantitative histological evaluation is produced according to the ISO 10993-6 standard method.

Histological micrographs are carried out for each rabbit.

Each analyzed parameter is noted according to the following scale:
0: Absent
1: Limited
2: Moderate
3: Pronounced/Few unincorporated “endoprostheses”
4: Very marked/severe/total/rupture or absence of limiting laminae.

These parameters make it possible to make a suitable evaluation of any reaction such as an inflammatory reaction, a reaction to a foreign body, an immunological reaction or a neointimal formation reaction.

The blind quantitative histomorphometric analysis is carried out as follows:

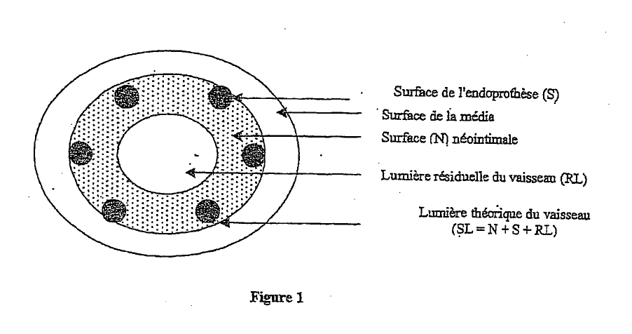

Each histological slide is read with a Zeiss™ axioscope-microscope that is equipped with 5×, 10×, 20×, and 40× magnifying lenses linked to a SAMBA™ image analyzer (SAMBA TECHNOLOGIES, France). The residual light of the vessel (RL) and the theoretical light of the vessel (SL) that corresponds to the surface under light delimited by the internal limiting lamina, the mean surface areas of the endoprosthesis (S) and the neointimal formation (N) are determined. FIG. 1 illustrates these definitions.

The mean thickness of the neointimal formation is measured, and the percentage of stenosis (P_{S}) of each arterial slide is calculated by the formula:

P_{S}=100×(S1+S2)/S

The mean percentage of reduction of the stenosis of group X relative to the group Y (P_{MR}) is calculated according to the following formula:

P_{MR}=100× (mean percentage of stenosis of group X—mean percentage of stenosis of group Y)/(mean percentage of stenosis of group X).

A statistical analysis by the non-parameterized Student's test at a 5% risk was achieved between the two groups.

### Results of the Histopathological Analysis

All of the arteries are of the muscular type. The endoprostheses are correctly deployed in the two groups. No physical event detrimental to the tests nor any visible microscopic physical degradation of the endoprostheses was observed.

The endoluminal surface area of all of the samples is completely doubled by endothelial-type cells.

### Group A

The structure of the endoprosthesis is well integrated in the neointimal tissue.

The thickening of the neointima is observed and is designated as “light” to “significant,” hence a mean score of 2 for the entire group.

In 19 of 20 slides, the neointima comprises cholesterol deposits in a designated magnitude of “light” to “moderate,” hence a mean score of 1.5, of macrophages, of spumous cells in a light to moderate proportion and several lymphocytes. For the last slide (identified under reference 11.1 FUO, left proximal), the layer of neointimal tissue has a moderate thickness and does not comprise a lipidic deposit.

The presence of a proteoglycan matrix in the neointimal tissue is suspected. These fatty cells and elements are covered by an endothelial fibrous layer with a moderate thickness that comprises a limited proportion of collagen.

Several vascular smooth muscle cells are observed now and then inside the neointimal tissue.

For 5 to 8 histological slides, a thinning referred to as “moderate” to “marked” of the media is observed, demonstrating a point rupture of the lamina or the absence of lamina. A protrusion of the endoprosthesis into the arterial wall is observed for two specimens with a serious injury in one of the cases (16.1 FYR right). No infection and no degradation of the adventitious coat is observed.

The histopathological reading of the slides of group A makes it possible to demonstrate—for nearly all of the samples—the presence of an arteriosclerotic plaque with a fibrous layer that results in a moderate arterial restenosis.

### Group B

The structure of the endoprosthesis is well integrated into the neointimal tissue.

The neointimal tissue that is developed in the samples of this group is generally thicker than for the group A, and its thickening is referred to as “light” to “very marked” according to the samples, hence a mean score of 3 for the entire group.

In all of the histological slides, the neointima comprises cholesterol deposits in a magnitude referred to as “light” to “marked,” hence a mean score of 2.5, of macrophages, of spumous cells in a moderate to marked proportion, and several lymphocytes.

The presence of a proteoglycan matrix in the neointimal tissue is suspected.

These fatty cells and elements that are observed in group B are of a more significant proportion than in the group A. These compounds are covered by an endothelial fibrous layer of moderate thickness, comprising a limited proportion of collagen.

New capillaries have been observed now and then inside the neointimal tissue. The cholesterol deposits, although in limited quantity, are more frequently observed in the group B than in the group A.

For 5 to 8 slides, a thinning that is referred to as “moderate” to “marked” of the media is observed, demonstrating a point rupture of the lamina or the absence of lamina. The media is sometimes infiltrated by fatty substances. For a sample (8.1 FUF, left proximal), calcification indices are observed in the media.

A protrusion of the endoprosthesis in the arterial wall is observed for two samples with a serious injury in one of the cases (5.1 FBZ left). Indications of an infection are noted in one of the cases (5.1 FBZ, medial left 2). No degradation of the adventitious coat is observed.

The histopathological reading of the slides of group B makes it possible to demonstrate, for nearly all the samples, the presence of an arteriosclerotic plaque with a fibrous layer that induces a much greater restriction of the light of the vessels than in the case of group A.

### Histomorphometric Analysis

The results of the histomorphometric analysis are set in Tables 2 to 4 below:

TABLE 2
Means (m) and Standard Deviations (d) of Each of the Determined Parameters
for the Groups A and B
Parameters: Surface of the implant: (S); Residual light of the vessel: (RL); Neointimal
surface: (N); Surface of the media: (M); Total surface: (T); Theoretical light of the
vessel (N + S + RL); Percentage of stenosis; Thickness of the neointima: E;

| | | | | | | | |
|---|---|---|---|---|---|---|---|
| S | RL | N | M | T | (N + S + RL) | Stenosis | E |
| (mm²) | (mm²) | (mm²) | (mm²) | (mm²) | (mm²) | (%) | (μm) |

Group A

| | | | | | | | |
|---|---|---|---|---|---|---|---|
| m: 0.20 | m: 3.45 | m: 1.07 | m: 0.22 | m: 4.94 | m: 4.72 | m: 28.1 | m: 178.8 |
| d: 0.06 | d: 1.00 | d: 0.37 | d: 0.10 | d: 1.15 | d: 1.11 | d: 9.5 | d: 61.8 |

Group B

| | | | | | | | |
|---|---|---|---|---|---|---|---|
| m: 0.21 | m: 2.87 | m: 1.78 | m: 0.27 | m: 5.12 | m: 4.86 | m: 42.2 | m: 315.0 |
| d: 0.07 | d: 1.04 | d: 0.67 | d: 0.13 | d: 0.79 | d: 0.75 | d: 15.4 | d: 152.8 |

TABLE 3
Mean Percentage of Reduction of the Intraprosthetic Restenosis

Intraprosthetic Restenosis
100 · (N + S)/(N + S + RL)

| Group A | Group B | Reduction of Stenosis |
|---|---|---|
| 28.1% | 42.2% | 33.4% |
| | | P = 0.0022 |

TABLE 4
Statistical Comparison (Student's Test at a 5% Risk)

| Statistical | | Thickness of the | Surface of the |
|---|---|---|---|
| Comparison | Stenosis (%) | Neointima (μm) | Neointima (mm²) |
| Group A Relative to | S | S | S |
| Group B | A < B | A < B | A < B |
| p | p = 0.0022 | p = 0.0014 | p = 0.0004 |

S: Significant Comparison

### Conclusion

The results that are obtained for the two groups (group A: group that is treated with the composition A; placebo group) demonstrate the following effects:

Nearly all of the instrumented arteries (40 of 41 histological sections) create the presence of an arteriosclerotic plaque with a fibrous layer associated with an intraprosthetic restenosis, moderate in the case of group A and marked in the case of group B.

The mean percentage of reduction of the endoprosthetic restenosis is 33.4% for the group A with a significant statistical difference (p=0.0022).

The initial arterial damages and the deployment of endoprostheses seem to be homogeneous for the two groups when the similar thicknesses of the media and the incidence of lesions of the media observed in the two groups are considered.

The composition (A) according to the invention therefore has a positive effect in the prevention of endoprosthetic restenosis.

### C) In-Vivo Study on the Activity of the Composition (A) on Arterial Pressure in Normotense Rats

The composition (A) given by oral administration for a week reduces the arterial pressure in nonmotense rats without modifying the cardiac frequency. The thermodynamic effects are combined with an improvement of the arterial vasodilation that depends on the endothelium and an induction of gene expression into the vascular wall that have the function of preserving the contractile response of the vessels.

The treatment by the composition (A) has a hypotensive effect in an experimental model of arterial hypertension. The treatment protects against cardiac fibrosis, aortic thickening, endothelial dysfunction and the increase of the contractile response in this model. These effects are combined with the increase of the NO-synthase activity both in the heart and in the aorta. These results demonstrate that the composition (A) protects against arterial hypertension and improve the cardiac and vascular functional and structural changes associated with this illness.

All of the results in the pharmacology of the composition (A) provide a scientific basis to the hypotheses resulting from epidemiological studies on its cardio-protective and vasculo-protective effects.

### D) Study on a Panel of Patients

One test is a double-blind versus placebo and is provided to determine the impact of supplementing the patients' diet with composition A on the frequency and the degree of restenosis in coronary patients who should benefit from an angioplasty with or without installation of (an) endoprosthesis (es), as well as the progression of atheromatous illness at the end of one year.

The panel of patients would be a population of about 300 patients that consist of the group of subjects (≧18 years) without gender distinction, addressed for the management of coronary heart disease and who should benefit from an angio-coronarography. Women of child-bearing age would be excluded from the panel. 50% of the panel would be treated with a placebo and 50% with the composition A.

The placebo group would be presumed to have a restenosis level of between 20% and 30%.

Taking into account the tests set forth in the preceding paragraph on rabbits, a positive result allowing the lowering of the restenosis level to a 15% maximum of the treated population or an improvement of 5% to 15% would be expected. The duration of the test would be 2.5 years with a minimum follow-up of the patients once per year.

1. Composition for the implementation of a method of preventive therapeutic treatment against endoprosthetic restenosis and against atheromatous illness in coronary patients, characterized in that it comprises between 60% and 80% by weight of the compounds of the flavonol family as active ingredient.

2. Composition as defined in claim 1, wherein it comprises, expressed in terms of percentage of flavonols that are present:
0% to 20% by weight of catechin and/or catechin-SH, 45% to 75% by weight of epicatechin and/or epicatechin-SH,
0% to 15% by weight of epicatechin gallate and/or epicatechin gallate-SH, and
15% to 25% by weight of epigallocatechin-SH.

3. Composition as defined in claim 1, wherein it also comprises between 10% and 30% by weight of compounds of the anthocyan family.

4. Composition as defined in claim 3, wherein it comprises, expressed in terms of percentage of the anthocyans that are present:
55% to 100% by weight of malvidin-3-O-glucoside, acetylated malvidin-3-O-glucoside and/or coumarylated malvidin-3-O-glucoside,
0% to 30% by weight of peonidin-3-O-glucoside, acetylated peonidin-3-O-glucoside and/or coumarylated peonidin-3-O-glucoside,
0% to 20% by weight of petunidin-3-O-glucoside and/or acetylated petunidin-3-O-glucoside,
0% to 10% by weight of cyanidin 3-O-gluoside and/or acetylated petunidin-3-O-glucoside, and
0% to 10% by weight of delphinidin 3-O-glucoside and/or acetylated delphinidin-3-O-glucoside.

5. Composition as defined in claim 1, wherein it also comprises between 10% and 30% by weight of compounds of the family of phenolic acids.

6. Composition as defined in claim 5, wherein it comprises, expressed in terms of percentage of phenolic acids that are present:
20% to 25% by weight of caftaric acid,
30% to 40% by weight of cafeic acid,
15% to 25% by weight of trans-coutaric acid,
15% to 25% by weight of para-coumaric acid,
optionally up to 5% of cis-coutaric acid, and
optionally up to 5% of GRP.

7. Composition as defined in claim 1, wherein it also comprises up to 5% of the compounds of the family of flavonols.

8. Composition as defined in claim 7, wherein it comprises, expressed in terms of percentage of flavonols that are present:
15% to 35% by weight of myricetol glucoside,
15% to 35% by weight of quercetol,
10% to 25% by weight of myricitol and
15% to 30% by weight of quercetol glucoside.

9. Composition as defined in claim 1, wherein it is obtained by a process that comprises the following successive stages:
A stage (a) for red wine distillation,
A stage (b) for concentration of the distillate that is obtained in stage (a), and
A stage (c) for drying the concentrate that is obtained in stage (b).

10. Application of a composition as defined in claim 1, for the implementation of a method for preventive therapeutic treatment against endoprosthetic restenosis and against atheromatous illness in coronary patients.

11. Composition as defined in claim 2, wherein it also comprises between 10% and 30% by weight of compounds of the anthocyan family.

12. Composition as defined in claim 3, wherein it comprises, expressed in terms of percentage of the anthocyans that are present:
55% to 100% by weight of malvidin-3-O-glucoside, acetylated malvidin-3-O-glucoside and/or coumarylated malvidin-3-O-glucoside,
0% to 30% by weight of peonidin-3-O-glucoside, acetylated peonidin-3-O-glucoside and/or coumarylated peonidin-3-O-glucoside,
0% to 20% by weight of petunidin-3-O-glucoside and/or acetylated petunidin-3-O-glucoside,
0% to 10% by weight of cyanidin 3-O-gluoside and/or acetylated petunidin-3-O-glucoside, and
0% to 10% by weight of delphinidin 3-O-glucoside and/or acetylated delphinidin-3-O-glucoside.

13. Composition as defined in claim 2, wherein it also comprises between 10% and 30% by weight of compounds of the family of phenolic acids.
